(19) United States
(12) Patent Application Publication
(10) Pub. No.: US 2018/0290162 A1
(43) Pub. Date: Oct. 11, 2018

(54) CONTAINER FOR FLUID PRODUCTS, PARTICULARLY PHARMACEUTICAL, COSMETIC, MEDICAL PRODUCTS OR THE LIKE

(71) Applicants: Jacopo Allamprese, Vedano al Lambro (IT); R BIO TRANSFER S.R.L., Albanella (IT)

(72) Inventor: Jacopo Allamprese, Vedano al Lambro (IT)

(21) Appl. No.: 15/542,366

(22) PCT Filed: Dec. 22, 2015
(86) PCT No.: PCT/IB2015/059888

§ 371 Date: Jul. 7, 2017

(30) Foreign Application Priority Data
Jan. 7, 2015 (IT) MO2015A000002

(51) Int. Cl. B05B 11/00; B65D 47/26; A45D 34/00; B05B 11/04; A61F 9/00

(52) U.S. Cl. CPC B05B 11/0078; B65D 47/265; A45D 34/00; A45D 2200/054; B05B 11/3081; A61F 9/0008; B05B 11/04

(57) ABSTRACT

A container for fluid products, particularly pharmaceutical, cosmetic, medical products or the like includes an hollow body divided into: a first tank containing a first fluid substance and communicating with a first dispensing hole of the first fluid substance; a second tank containing a second fluid substance and communicating with a second dispensing hole of the second fluid substance; a neck associated with the hollow body and on which are formed the first dispensing hole and the second dispensing hole; a selector element associated with the neck by interposition of rotation means able to rotate the selector element around an axis of rotation, and the selector element includes an outlet channel able to dispense selectively, due to the rotation of the selector element, the first fluid substance or the second fluid substance.

### TECHNICAL FIELD

The present invention relates to a container for fluid products, particularly pharmaceutical, cosmetic, medical products or the like.

### BACKGROUND ART

With reference to the field of eye drops and solutions for ophthalmic use it is known that some of these products only include one active ingredient, or two or more active ingredients combined together chemically.

Clinical practice often requires, however, especially in the treatment of more complex diseases, the use of different eye drops, each contained in a single container, to instill in rapid succession or in combination thereof several times a day.

In addition, patients suffering from complex diseases, such as glaucoma, have to protract the treatment for several years and, at times, for a lifetime.

In this regard, it is appropriate to note that the instillation of eye drops is usually performed by the patient him/herself and implies the opening of each bottle, the administration of the eye drops, the closure of the bottle and the repetition of this procedure for each eye drop prescribed in the treatment.

It is easy to understand how the above procedure has long execution times and proves difficult, especially if repeated for several years, leading to a marked decrease in the perseverance of the patient in following the treatment and, therefore, to a subsequent worsening of the pathological aspect.

Moreover, if the eye drops contain several active ingredients the latter are not always chemically miscible with each other.

In the case e.g. of a water-based substance and of a fatty-based substance their physical-chemical differences would lead them to float over one another, thus preventing the eye drops from mixing properly and, therefore, the correct dosage of the latter.

To obviate at least in part to these problems, today containers are used having two containment chambers mutually separate and not communicating able to contain different drug solutions and to allow for the simultaneous dispensing of both solutions, as is known from the patent document TO2011A000495.

Such containers, however, have several drawbacks among which must be counted the fact that the two drug solutions may not mix properly, causing uncertainties of dosage, or may give rise to substances not acceptable in therapy.

### DESCRIPTION OF THE INVENTION

The main aim of the present invention is to provide a container for fluid products, particularly pharmaceutical, cosmetic, medical products or the like which allows rapid administration in sequence, reducing the number of installations to be performed and, therefore, considerably decreasing the tuning of execution of such medical practice.

One object of the present invention is to provide a container for fluid products, particularly pharmaceutical, cosmetic, medical products or the like which allows to reduce the number of containers to be used, facilitating the administration procedure and facilitating the perseverance of the patient in therapy.

Another object of the present invention is to provide a container for fluid products, particularly pharmaceutical, cosmetic, medical products or the like which allows to combine, in a single container, different eye drops or other type of fluid products according to the patient's needs.

A further object of the present invention is to provide a container for fluid products, particularly pharmaceutical, cosmetic, medical products or the like which allows to overcome the mentioned drawbacks of the prior art within the ambit of a simple, rational, easy, effective to use and affordable solution.

The objects stated above are achieved by the present container for fluid products, particularly pharmaceutical, cosmetic, medical products or the like having the characteristics of claim 1.

### BRIEF DESCRIPTION OF THE DRAWINGS

Other characteristics and advantages of the present invention will become better evident from the description of a preferred, but not exclusive, embodiment of a container for fluid products, particularly pharmaceutical, cosmetic, medical products or the like, illustrated by way of an indicative but non-limiting example, in the accompanying drawings in which:

FIG. 1 is an exploded view of a container according to the invention;

FIG. 2 is an exploded view, from another angle, of a container according to the invention;

FIG. 3 is an axonometric view of the container according to the invention;

FIG. 4 is a sectional front view of the container according to the invention in a first operating configuration;

FIG. 5 is a sectional front view of the container according to the invention in a second operating configuration.

### EMBODIMENTS OF THE INVENTION

With particular reference to such figures, reference number 1 generally designates a container for fluid products, particularly pharmaceutical, cosmetic, medical products or the like.

In this regard it is specified that in this treatise by the term fluid products is not only intended liquid products but also viscous products, e.g. in the state of paste and gel, and powder products, in particular very fine powders with great smoothness.

The container 1 comprises a hollow body 2 having a substantially tubular and elongated conformation and a bottom 3.

Usefully, with reference to the normal resting position of the container 1 it is also possible to locate a substantially vertical longitudinal axis A.

In this sense it is specified that in the context of this specification expressions such as “top”, “at the top”, “on top” “bottom”, “upper”, “lower”, “above”, “below” and the like, are to be considered with reference to the above resting position, wherein the container 1 is placed with the bottom 3 resting on a substantially horizontal surface.

The hollow body 2 is divided into a first tank 4a, containing a first fluid substance 5a and communicating with a first dispensing hole 6 of the first fluid substance 5a, and into a second tank 4b, containing a second fluid substance 5b and communicating with a second dispensing hole 7 of the second fluid substance 5b.

In the particular embodiment shown in the figures, the first tank 4a and the second tank 4b are composed of two distinct and separate compartments associable with each other.

In detail, each tank 4a, 4b has a substantially semi-cylindrical shape with a flat face 8a, 8b and a curved face 9a, 9b.

When the hollow body 2 is assembled, the two tanks 4a, 4b are coupled to the flat faces 8a, 8b substantially mating and juxtaposed, and the resulting hollow body 2 is shaped like a cylinder.

In addition, each tank 4a, 4b comprises a closed portion 10a, 10b, arranged at the base of the corresponding faces 8a, 9a and 8b, 9b, and an open portion 11a, 11b, arranged at the top of the corresponding faces 8a, 8b and 9a, 9b. The closed portions 10a, 10b are able to define the bottom 3 of the hollow body 2.

The open portions 11a, 11b are able to define a narrowing 12a, 12b, having a diameter less than the rest of the tank 4a, 4b.

Moreover, the first tank 4a and the second tank 4b are made at least partly of an elastically deformable material.

The first fluid substance 5a or the second fluid substance 5b can be dispensed due to the pressure applied on one of the curved faces 9a, 9b.

In particular, by the expression “elastically deformable material” is meant a material with flexibility and elasticity such as to allow the user to dispense the first fluid substance 5a or the second fluid substance 5b by means of the pressure applied on the predetermined curved face 9a, 9b; in other words, by “elastically deformable material” is meant a material able to deform in a reversible manner as a result of external stresses.

The first tank 4a and the second tank 4b may have different colors and/or symbols depending on the user's needs.

In particular, these colors and/or symbols are designed to help the user to identify and memorize the first fluid substance 5a and the second fluid substance 5b or, alternatively, to differentiate the different administrations over the day.

Alternative embodiments cannot, however, be ruled out wherein the hollow body 2 is composed of a single monolithic body having an internal separation wall able to delimit the first tank 4a and the second tank 4b.

The container 1 comprises a neck 13 associated with the hollow body 2 and on which are formed the first dispensing hole 6 and the second dispensing hole 7.

The neck 13 has a substantially tubular and elongated shape along the longitudinal axis A.

In the present case, the first dispensing hole 6 and the second dispensing hole 7 are arranged on top of the neck 13.

In the embodiment shown in the figures, the neck 13 and the hollow body 2 are associable with each other by interposition of the coupling means 14.

In detail, the coupling means 14 comprise a ring element, formed inside the neck 13 and associable by interlocking with the narrowing 12a, 12b of each tank 4a, 4b.

From the ring element 14 depart a first dispensing duct 15 and a second dispensing duct 16.

Each dispensing duct 15, 16 comprises a lower opening, which in the assembly configuration, mates with the open portion 11a, 11b of each tank 4a, 4b and an upper opening, which defines the dispensing holes 6, 7.

The dispensing ducts 15, 16 extend longitudinally and substantially parallel to the longitudinal axis A.

Advantageously, the dispensing ducts 15, 16 have a decreasing diameter along the longitudinal axis A, larger in the proximity of the lower opening and narrower in the proximity of the upper opening.

It cannot be ruled out, however, that the hollow body 2 and the neck 13 may be composed of a single monolithic body.

In this case the hollow body 2 extends into the neck 13 and has dispensing holes 6, 7 through which the fluid substances 5a, 5b can be made to exit and applied by the user.

The container 1 comprises a selector element 17 associated with the neck 13 by interposition of rotation means 18, 19 and comprising an outlet channel 20 able to dispense selectively, due to the rotation of the selector element itself, the first fluid substance 5a and the second fluid substance 5b.

With reference to the particular embodiment shown in the figures, the selector element 17 is cap-shaped and can be fitted around the neck 13.

However, it cannot be ruled out that the selector element 17 can take different shapes, that is, e.g., that it is made in the form of a flat surface rotatable on top of the neck 13.

The selector element 17 is associated in a sliding manner on the neck 13.

In this specification, the fact that the selector element 17 slides in rotation on the neck 13 means that they are restrained so as to define a generic kinematic pair, in this case rotational, whose freedom of movement allows the rotation of the selector element itself on the neck 13.

In other words, the selector element 17 and the neck 13 are locked so as not to allow any relative translational movement, but only rotational movements.

The rotation means 18, 19 are able to rotate the selector element 17 around an axis of rotation R which, with reference to the particular embodiment shown in the figures, coincides with the longitudinal axis A.

The rotation means 18, 19 are able to rotate the selector element 17 between a first operating configuration, shown in FIG. 4, wherein the outlet channel 20 is substantially mating with the first dispensing hole 6, and a second operating configuration, shown in FIG. 5, wherein the outlet channel 20 is substantially mating with the second dispensing hole 7.

Advantageously, the rotation means 18, 19 comprise a groove 18 formed on at least one of the neck 13 and the selector element 17 and an abutment protrusion 19 formed on the other of the neck 13 and the selector element 17 and at least partly inserted into the groove 18.

With reference to the particular embodiment shown in the figures, the groove 18 is formed on the neck 13 and the abutment protrusion 19 is formed on the selector element 17.

It cannot be ruled out, however, that the groove 18 can be formed on the selector element 17 and the abutment protrusion 19 can be formed on the neck 13.

Conveniently, the groove 18 has a substantially annular shape, i.e. has an angular extension substantially equal to 360′; this means that it entirely surrounds the entire perimeter of the neck 13.

In the longitudinal direction, on the other hand, the groove 18 extends between a first shoulder 21 and a second shoulder 22.

The first shoulder 21 is arranged above the second shoulder 22, which in turn is located in the proximity of the tanks 4a, 4b.

Advantageously, similarly to the groove 18, the abutment protrusion 19 also has a substantially annular conformation but, in the longitudinal direction, has an extension lower than the groove 18.

The abutment protrusion 19 is in rotational sliding contact on the first shoulder 21 and, in addition to ensuring the rotation of the selector element 17 around the neck 13, it also performs the function of preventing the selector element 17 from removing and the separation thereof from the neck 13.

It is underlined, however, that alternatives embodiments of the present invention are possible wherein the groove 18 and the abutment protrusion 19 have different shapes.

It is possible, in fact, that the groove 18 has an angular extension lower than 360°, e.g. substantially equal to 180°, and the abutment protrusion 19 consists of a centripetal pin.

It is easy to understand that this conformation allows to move the selector element 17 between two diametrically opposite positions by making it rotate back and forth in both directions of rotation.

The container 1 comprises temporary locking means 23, 24 in the first operating configuration and in the second operating configuration.

In other words, the temporary locking means 23, 24 are able to keep the selector element 17 in the first operating configuration (FIG. 4) and in the second operating configuration (FIG. 5).

Advantageously, the temporary locking means 23, 24 comprise at least one tooth 23, formed on at least one of the neck 13 and the selector element 17, and at least two fitting seats 24, formed on the other of the neck 13 and the selector element 17 and wherein the tooth itself is alternately insertable.

In the context of this specification the term “alternately” refers to the fact that the tooth 23 can be inserted into a fitting seat 24, so as to lock the selector element 17 in the first operating configuration, or in the other fitting seat 24, so as to lock the selector element 17 in the second operating configuration.

It is useful to point out that with tooth 23 is defined a protuberance formed inside the selector element 17 and able to be inserted, locking the selector element itself, in the fitting seats 24.

Advantageously, the tooth 23 is formed on the selector element 17 and the fitting seats 24 are formed on the neck 13; it cannot be ruled out, however, that the tooth 23 is formed on the neck 13 and the fitting seats 24 are formed on the selector element 17.

With reference to the particular embodiment shown in the figures, the selector element 17 comprises two teeth 23 and two fitting seats 24.

The teeth 23 and the fitting seats 24 are arranged diametrically opposite, that is, staggered by 180° with respect to the axis of rotation R.

When a tooth 23 is inserted into a fitting seat 24, the other tooth 23 is inserted in the other fitting seat 24, and vice versa.

It is emphasized, however, that the number of teeth 23 and of fitting seats 24 is variable depending on the number of tanks 4a, 4b present in the hollow body 2. In other words, it cannot be ruled out that the hollow body 2 can be divided into three or more tanks 4a, 4b, each containing a different fluid substance, in which case it is necessary that the temporary locking means 23, 24 provide the same number of fitting seats 24.

The disengagement of the teeth 23 from the fitting seats 24 is obtained by exploiting the deformability of the walls of at least one of the neck 13 and the selector element 17.

In this regard it is underlined that the selector element 17 comprises at least one longitudinal split 25 able to facilitate the rotation of the selector element itself. With reference to the embodiment shown in the figures, two longitudinal splits 25 are provided which divide the selector element 17 into two mutually opposite flaps 26 on each of which a corresponding tooth 23 is arranged.

The longitudinal splits 25, in practice, are able to allow the partial deformability in the centripetal direction of the selector element 17.

To facilitate the start of the rotation, the selector element 17 may comprise gripping means of the type one or more reliefs, a knurled surface, or the like.

The outlet channel 20 comprises a first open extremity 27 arranged on top of the selector element 17 and a second open extremity 28, coinciding with the first dispensing hole 6 or with the second dispensing hole 7.

In detail, the first open extremity 27 lies on the axis of rotation R.

It cannot be ruled out, however, that the first open extremity 27 can be moved, i.e., not coincident with the axis of rotation R.

With reference to the particular embodiment shown in the illustrations, the outlet channel 20 extends along a direction substantially inclined with respect to the axis of rotation R.

In other words, the outlet channel 20 extends obliquely with respect to the axis of rotation R, that is, neither vertically nor horizontally. In detail, the substantially inclined direction of the outlet channel 20 avoids the permanence inside it of any residue of the first fluid substance 5a or of the second fluid substance 5b.

However, in an alternative embodiment not shown in the figures, the outlet channel 20 extends in a direction substantially parallel to the axis of rotation R. It is easy to understand how in this case the first open extremity 27 and the second open extremity 28 are arranged vertically aligned with one another.

The container 1 comprises a removable covering element 29 which is associable with the neck 13 to cover the selector element 17.

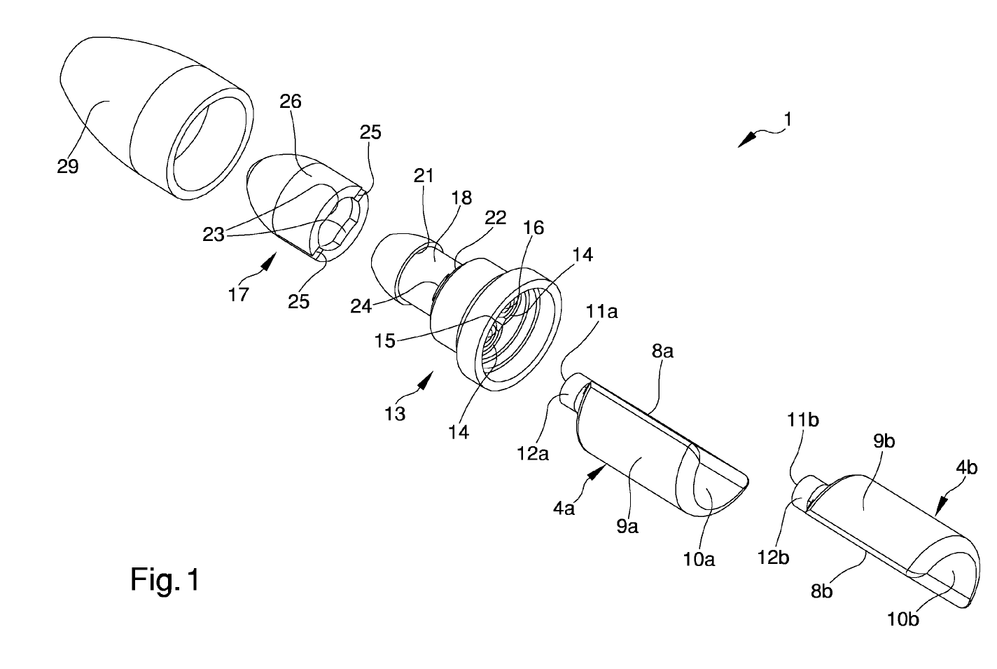

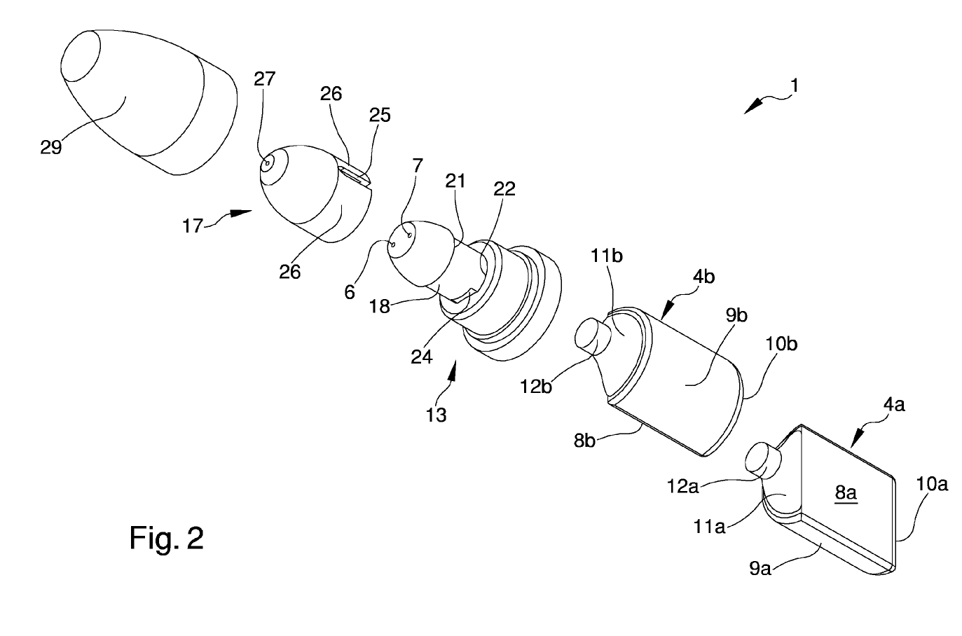

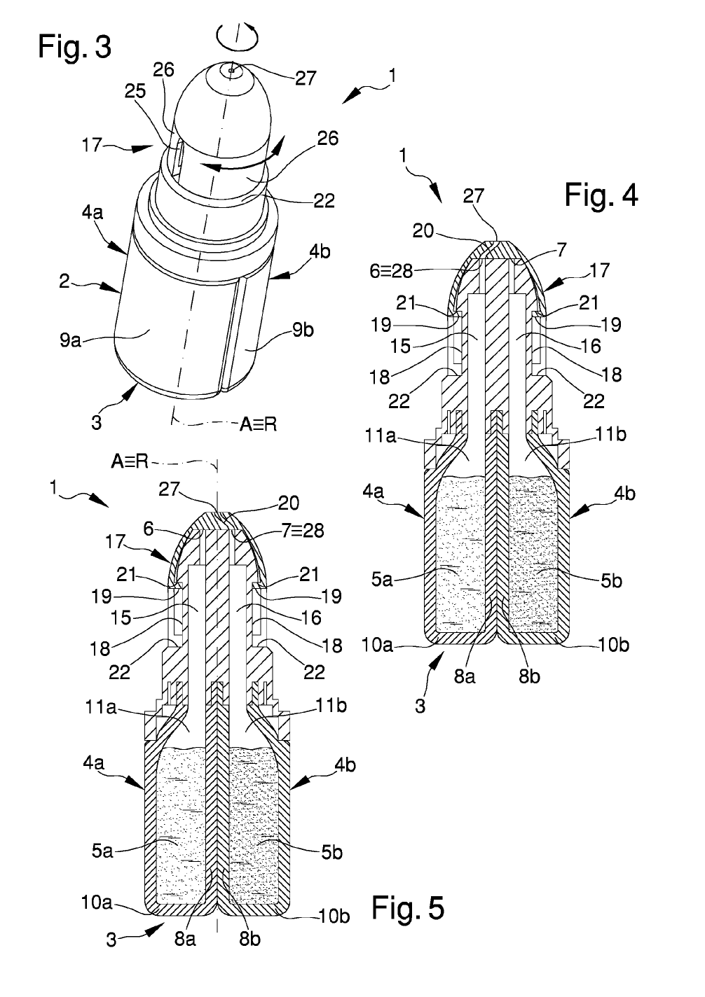

With reference to FIGS. 1 and 2, the covering element 29 is shown in the form of a traditional cap normally in use in containers for eye drops.

Advantageously, the cap 29 has a shape substantially mating with the selector element 17, in the present case cap-like.

It should be stressed that the presence of the cap 29 ensures the isolation of the first fluid substance 5a and of the second fluid substance 5b, thus considerably reducing the risk of environmental contamination.

The operation of the present invention is as follows.

The cap 29 is removed upon use and according to the user's needs the selector element 17 is rotated towards the first tank 4a or the second tank 4b.

In the first case, the outlet channel 20 is positioned so as to communicate with the first dispensing hole 6 to allow the administration of the first fluid substance 5a (first operating configuration of FIG. 4).

In detail, the first fluid substance 5a flows out from the outlet channel 20 due to the pressure applied by the user on the curved wall 9a.

In the second case, the outlet channel 20 is positioned so as to communicate with the second dispensing hole 7 to allow the administration of the second fluid substance 5b (second operating configuration of FIG. 5).

Similarly to the first operating configuration of FIG. 4, the second fluid substance 5b flows out from the outlet channel 20 due to the pressure applied by the user on the second curved wall 9b.

In both operating configurations, the temporary locking means 23, 24 retain the selector element 17, in the first operating configuration or in the second operating configuration, for the entire duration of the dispensing.

It is underlined that the particular solution of providing two separate tanks and one selector element allows the selection and selective dispensing of the fluid substances contained in the tanks, thus preventing mutual contamination between the latter.

Indeed, the presence of the selector element allows to dispense one single fluid substance at a time, without mixing in any way the latter with the fluid substance contained in the other tank.

Added to this is the fact that the flexibility of the tanks together with the reduced diameter of the outlet channel allows precise dispensing.

1. A container for fluid products, particularly pharmaceutical, cosmetic, medical products or the like, comprising:
at least a hollow body divided into:
at least a first tank containing a first fluid substance and communicating with a first dispensing hole of said first fluid substance;
at least a second tank containing a second fluid substance and communicating with a second dispensing hole of said second fluid substance;
at least a neck associated with said hollow body and on which are formed said first dispensing hole and said second dispensing hole;
wherein said container (1) comprises at least a selector element associated with said neck by interposition of rotation means able to rotate said selector element around an axis of rotation, said selector element comprising at least an outlet channel able to dispense selectively, due to the rotation of said selector element, said first fluid substance or said second fluid substance.

2. The container according to claim 1, wherein said rotation means are able to rotate said selector element between a first operating configuration wherein said outlet channel is substantially mating with said first dispensing hole and a second operating configuration wherein said outlet channel is substantially mating with said second dispensing hole.

3. The container according to claim 1, wherein said rotation means comprise at least a groove formed on at least one of said neck and said selector element and at least an abutment protrusion formed on the other of said neck and said selector element and at least partly inserted into said groove.

4. The container according to claim 3, wherein said groove is formed on said neck and said abutment protrusion is formed on said selector element.

5. The container according to claim 3, wherein said groove has a substantially annular shape.

6. The container according to claim 3, wherein said groove extends between a first shoulder and a second shoulder.

7. The container according to claim 3, wherein said abutment protrusion has a substantially annular shape.

8. The container according to claim 6, wherein said abutment protrusion is in rotational sliding contact on said first shoulder.

9. The container according to claim 2, wherein said container (1) comprises temporary locking means in said first operating configuration and in said second operating configuration.

10. The container according to claim 9, wherein said temporary locking means comprise at least a tooth formed on at least one of said neck and said selector element and at least two fitting seats formed on the other of said neck and said selector element and wherein said tooth is alternately insertable.

11. The container according to claim 10, wherein said tooth is formed on said selector element and said fitting seats are formed on said neck.

12. The container according to claim 1, wherein said selector element is cap-shaped and can be fitted around said neck.

13. The container according to claim 1, wherein said outlet channel comprises a first open extremity arranged on top of said selector element and a second open extremity coinciding with said first dispensing hole or with said second dispensing hole.

14. The container according to claim 13, wherein said first open extremity lies on said axis of rotation.

15. The container according to claim 1, wherein said outlet channel extends along a direction substantially inclined with respect to said axis of rotation.

16. The container according to claim 1, wherein said first tank and said second tank have a substantially semi-cylindrical shape.

17. The container according to claim 16, wherein said first tank and said second tank have a flat face and a curved face respectively, said tanks being assembled together with said flat faces substantially mating and juxtaposed to define said hollow body with a substantially cylindrical shape.

18. The container according to claim 1, wherein said first tank and said second tank are made at least partly of an elastically deformable material, said first fluid substance or said second fluid substance being dispensable due to the pressure applied on one of said curved faces.
